(19) United States
(12) Patent Application Publication
(10) Pub. No.: US 2026/0335568 A1
(43) Pub. Date: Oct. 29, 2026

(54) POWDER DELIVERY DEVICES AND METHODS OF USE

(71) Applicant: Boston Scientific Scimed, Inc., Maple Grove, MN (US)

(72) Inventors: Henry STOCK, Boston, MA (US); John Joseph SUBASIC, Sutton, MA (US); Narunn SUON, Lawrence, MA (US)

(73) Assignee: Boston Scientific Scimed, Inc., Maple Grove, MN (US)

(21) Appl. No.: 19/656,467

(22) Filed: Apr. 23, 2026

Related U.S. Application Data
(60) Provisional application No. 63/795,758, filed on Apr. 28, 2025.

(51) Int. Cl. A61M 13/00; A61M 39/22

(52) U.S. Cl. CPC A61M 13/00; A61M 39/22; A61M 2202/064

(57) ABSTRACT

A device for delivering an agent that includes an enclosure configured to store the agent and a channel configured to receive a pressurized fluid. The channel bypasses the enclosure. The device includes a first valve disposed downstream from the enclosure and the channel, and a second valve disposed upstream from the enclosure and the channel. The first valve is configured to selectively direct the pressurized fluid towards at least one of the enclosure and the channel. The second valve is configured to selectively direct the pressurized fluid received from the channel and the agent received from the enclosure out of the device for delivery. The first and second valves are configured to move simultaneously between a plurality of positions for selectively directing the pressurized fluid through the device via each of the channel, the enclosure, and both of the channel and the enclosure.

### CROSS-REFERENCE TO RELATED APPLICATION(S)

This application claims the benefit of priority to U.S. Provisional Application No. 63/795,758, filed on Apr. 28, 2025, which is incorporated by reference herein in its entirety.

### TECHNICAL FIELD

Various embodiments of the present disclosure relate generally to devices and methods for delivering agents. More specifically, this disclosure relates to devices for delivery of powdered agents, such as prophylactic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines. During an endoscopic procedure, a user inserts a sheath of an endoscope into a body lumen of a patient. The user utilizes a handle of the endoscope to control the endoscope during the procedure. Tools may be passed through a working channel of the endoscope via, for example, a port in the handle, to deliver treatment at the procedure site near a distal end of the endoscope. The procedure site is remote from the user.

To achieve hemostasis at the remote site, an agent (e.g., a prophylactic bio-adhesive) may be delivered by a delivery device inserted into the working channel of the endoscope. Agent delivery may be achieved, for example, through mechanical systems. Such systems, however, may require numerous steps or actuations to achieve delivery, may not achieve a desired rate of agent delivery or a desired dosage of agent, may result in the agent clogging portions of the delivery device, may result in inconsistent dosing of the agent, and/or may not result in the agent reaching the treatment site deep within the gastrointestinal tract. The current disclosure may solve one or more of these issues or other issues in the art.

### SUMMARY OF THE DISCLOSURE

Aspects of the disclosure relate to, among other things, systems, devices, and methods for a medical device including a valve assembly for delivering an agent (e.g., a prophylactic powder, a hemostatic powder, a bio-adhesive, etc.). More specifically, this disclosure relates to devices for delivery of powdered agents, such as prophylactic agents, via a valve assembly that includes an enclosure for storing the prophylactic agent and one or more valves that are movably disposed inside the device to facilitate selective delivery of a pressurized fluid and the prophylactic agent out of the device. Each of the aspects disclosed herein may include one or more of the features described in connection with any of the other disclosed aspects.

According to an example, a device for delivering an agent comprises an enclosure configured to store the agent; a channel configured to receive a pressurized fluid, wherein the channel bypasses the enclosure; a first valve disposed downstream from the enclosure and the channel, wherein the first valve is configured to selectively direct the pressurized fluid towards at least one of the enclosure and the channel; and a second valve disposed upstream from the enclosure and the channel, wherein the second valve is configured to selectively direct the pressurized fluid received from the channel and the agent received from the enclosure out of the device for delivery; wherein the first valve and the second valve are configured to move simultaneously between a plurality of positions for selectively directing the pressurized fluid through the device via each of the channel, the enclosure, and both of the channel and the enclosure.

Any of the devices described herein may include any of the following features. An inlet in fluid communication with a fluid source that is configured to store the pressurized fluid; and an outlet in fluid communication with a delivery conduit. The enclosure includes a first opening in fluid communication with the inlet and a second opening in fluid communication with the outlet. The channel includes a first end positioned adjacent to the first opening and in fluid communication with the inlet, and a second end positioned adjacent to the second opening and in fluid communication with the outlet. When in a first position, the first valve is configured to close the first opening of the enclosure and open the first end of the channel while the second valve is configured to close the second opening of the enclosure and open the second end of the channel. When in the first position, the first valve and the second valve are configured to fluidly decouple the enclosure from the inlet and the outlet, such that the first valve is configured to inhibit the pressurized fluid from entering the enclosure and the second valve is configured to inhibit the agent from exiting the enclosure. When in the first position, the first valve and the second valve are configured to fluidly couple the channel with the inlet and the outlet, such that the first valve is configured to allow the pressurized fluid to enter the channel and the second valve is configured to allow the pressurized fluid to exit the channel. When in a second position, the first valve is configured to open the first opening of the enclosure and close the first end of the channel while the second valve is configured to open the second opening of the enclosure and close the second end of the channel. When in the second position, the first valve and the second valve are configured to fluidly couple the enclosure with the inlet and the outlet, such that the first valve is configured to allow the pressurized fluid to enter the enclosure and the second valve is configured to allow a mixture of the agent and the pressurized fluid to exit the enclosure. When in the second position, the first valve and the second valve are configured to fluidly decouple the channel from the inlet and the outlet, such that the first valve is configured to inhibit the pressurized fluid from entering the channel and the second valve is configured to inhibit a remainder of the pressurized fluid from exiting the channel. When in a third position, the first valve is configured to open the first opening of the enclosure and the first end of the channel, and the second valve is configured to open the second opening of the enclosure and the second end of the channel. When in the third position, the first valve and the second valve are configured to fluidly couple each of the enclosure and the channel with the inlet and the outlet, such that the first valve is configured to allow the pressurized fluid to enter the enclosure and the channel while the second valve is configured to allow a mixture of the agent and the pressurized fluid to exit the enclosure and the pressurized fluid from the channel to deliver the mixture towards the outlet. When in a first position, the first valve is configured to direct the pressurized fluid into the channel and away from the enclosure while the second valve is configured to direct the pressurized fluid out of the channel and away from the enclosure, such that the pressurized fluid bypasses the enclosure for delivery in isolation from the agent. When in a second position, the first valve is configured to direct the pressurized fluid into the enclosure and away from the channel, thereby agitating the agent stored within the enclosure with the pressurized fluid. When in the second position, the first valve is configured to generate a mixture of the pressurized fluid and the agent within the enclosure while the second valve is configured to release the mixture out of the enclosure.

According to another example, a device for delivering an agent comprises an enclosure configured to store the agent; a first channel configured to receive a first portion of a pressurized fluid; a second channel configured to receive a second portion of the pressurized fluid, wherein the second channel is in fluid communication with the enclosure such that the second channel is configured to deliver the second portion of the pressurized fluid into the enclosure for mixing with the agent; a first valve disposed within the first channel and downstream from the enclosure, wherein the first valve is configured to selectively direct the first portion of the pressurized fluid received in the first channel out of the device for delivery; and a second valve disposed downstream from the enclosure and upstream from the first channel, wherein the second valve is configured to selectively direct the second portion of the pressurized fluid received from the second channel and the agent received from the enclosure into the first channel; wherein the first valve and the second valve are configured to move simultaneously between a plurality of positions for selectively directing the pressurized fluid through the device via one or more of the first channel, the second channel, and the enclosure.

Any of the devices described herein may include any of the following features. When in a first position, the first valve is configured to allow the first portion of the pressurized fluid received in the first channel to exit the device for delivery while the second valve is configured to inhibit a mixture of the agent and the second portion of the pressurized fluid received in the enclosure from the second channel from entering the first channel. When in a second position, the first valve is configured to allow the mixture to enter the first channel and the first portion of the pressurized fluid to direct the mixture out of the first channel out of the device for delivery.

According to a further example, a device for delivering an agent comprises an enclosure configured to store the agent; a channel configured to receive a pressurized fluid, wherein the channel bypasses the enclosure; a first valve disposed upstream from the enclosure and the channel, wherein the first valve is configured to selectively direct the pressurized fluid towards at least one of the enclosure and the channel; and a second valve disposed downstream from the enclosure, wherein the second valve is configured to selectively direct the agent out of the enclosure and into the channel; wherein the first valve and the second valve are configured to move simultaneously between a plurality of positions for selectively directing the pressurized fluid through the device via each of the channel and the enclosure.

Any of the devices described herein may include any of the following features. When in a first position, the first valve is configured to allow the pressurized fluid to enter the channel and exit the device for delivery while the second valve is configured to inhibit the agent from exiting the enclosure into the channel; and wherein in a second position, the first valve is configured to allow the pressurized fluid to enter the enclosure to generate a mixture with the agent while the second valve is configured to allow the mixture to exit the enclosure into the channel for delivery out of the device.

It may be understood that both the foregoing general description and the following detailed description are exemplary and explanatory only and are not restrictive of the invention, as claimed. As used herein, the terms “comprises,” “comprising,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “exemplary” is used in the sense of “example,” rather than “ideal.” The term “approximately,” or like terms (e.g., “substantially”), includes values +/- 10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device, according to one or more embodiments.

FIG. 2 shows a partial side view of an exemplary valve assembly of the delivery device of FIG. 1, according to one or more embodiments.

FIG. 3 shows a partial side view of another exemplary valve assembly of delivery device of FIG. 1, according to one or more embodiments.

FIG. 4 shows a partial side view of another exemplary valve assembly of delivery device of FIG. 1, according to one or more embodiments.

### DETAILED DESCRIPTION OF EMBODIMENTS

Embodiments of this disclosure relate to dispensing devices having valve assemblies for releasing a pressurized fluid (e.g., gas, air, liquid, etc.) and a material (e.g., a powdered agent) to a site of a medical procedure. In other words, the valve assemblies may be configured and operable to move stagnant material and facilitate proper dispensing of the material via the pressurized fluid. The valve assembly may include one or more valves movably disposed inside device for selectively controlling delivery of a pressurized fluid and agent stored in an enclosure, in order to combine the agent and pressurized fluid. The enclosure may store a predetermined amount of the agent, and may be in fluid communication with a pressurized fluid medium source storing the pressurized fluid. Upon release of the pressurized fluid into the enclosure by the one or more valves, the agent and the fluid may be combined within the enclosure prior to delivery from the device.

The pressurized fluid may be delivered through the valve assembly while diverting around the enclosure via the one or more valves of the valve assembly to deliver the pressurized fluid in isolation without the agent. The agent stored in the enclosure may be placed in fluid communication with the pressurized fluid through at least one of the one or more valves of the valve assembly. Accordingly, when the agent is combined with pressurized fluid, the valve assembly delivers the combination of the agent and the pressurized fluid through an outlet conduit to a target treatment site during a medical procedure. Aspects of the dispensing device and valve assembly, such as the one or more valves, may facilitate agitation and combination of the agent and the pressurized fluid prior to dispensing to prevent or minimize clogging of the dispensing device during delivery. The valve assembly may be configured and operable to move the agent that may be stored in a stagnant state within the enclosure and facilitate proper dispensing of the agent out of the dispensing device.

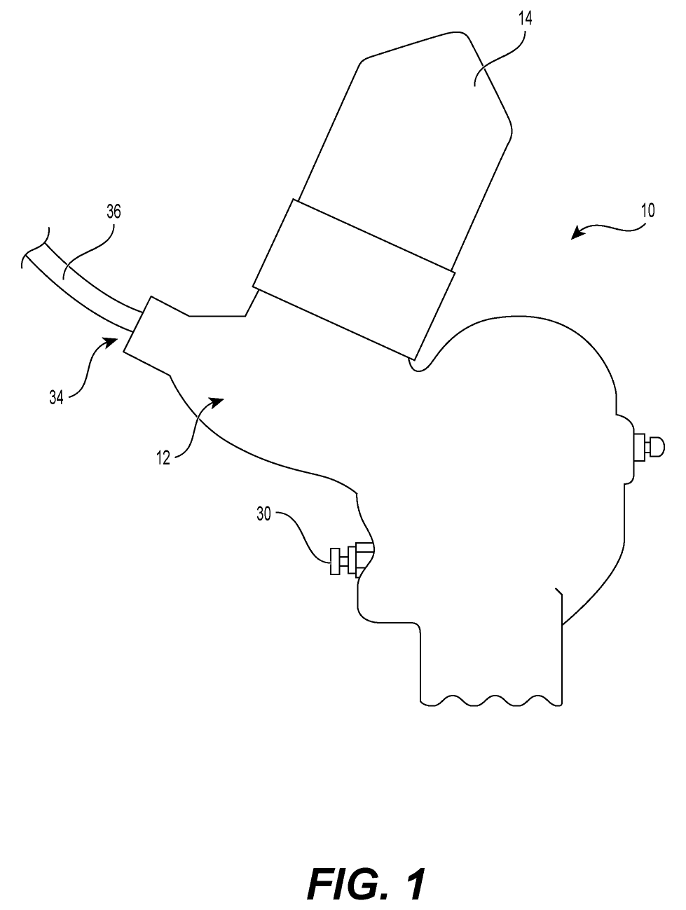

FIG. 1 shows a delivery system 10, which may be a powder delivery system. Delivery system 10 may include a handle body 12. Handle body 12 may include, or may be configured to receive, an enclosure 14 (or other source or container) storing a material (e.g., a powdered agent). Enclosure 14 may be coupled to handle body 12 for providing the agent to handle body 12, or a lid/enclosure of the agent may be screwed onto, or otherwise coupled to, enclosure 14 for supplying the agent to enclosure 14. The agent may be, for example, a powdered agent, such as a prophylactic agent, a hemostatic agent, or other various agents. In some examples, the prophylactic agent may include a bio-adhesive operable to promote natural healing by stopping a bleeding upon application to the target treatment site. Additionally, the prophylactic agent may be operable to generate a protective covering of the target treatment site experiencing the bleeding to mitigate subsequent rebleeding occurrences. In some embodiments, the powdered agent may include a particle size of less than about 100 µm. The agent may alternatively be another type of agent or material, or form of agent (e.g., a liquid or gel agent), and may have any desired function. Enclosure 14 may be removably attached to other components of delivery system 10, including components of handle body 12.

Handle body 12 may have a variety of features, to be discussed in further detail herein. U.S. Patent Application No. 16/589,633, filed Oct. 1, 2019, published as U.S. Patent No. 11,642,281 B1 on May 9, 2023, the disclosure of which is hereby incorporated by reference in its entirety, discloses features of exemplary delivery devices and systems. The features of this disclosure may be combined with any of the features described in the above-referenced patent. The features described herein may be used alone or in combination and are not mutually exclusive. Like reference numbers and/or terminology are used to denote similar structures, when possible.

Still referring to FIG. 1, delivery system 10 may include an actuation mechanism 30 used to activate flow of a pressurized fluid (e.g., gas, air, liquid, etc.) from a pressurized fluid source (not shown) in fluid communication with delivery system 10. In some examples, the pressurized fluid source may be external to delivery system 10. In other examples, the pressurized fluid source (e.g., gas or air canister) may be disposed inside delivery system 10, such as internal to handle body 12. Actuation mechanism 30 may be selectively actuated (e.g., manually depressible) or otherwise moved or actuated to control delivery of a material (e.g., a powdered agent) and/or the pressurized fluid. The pressurized fluid alone, or a combination of a powdered agent and the pressurized fluid, may be delivered from an outlet 34 of handle body 12. Outlet 34 may be in fluid communication with a delivery conduit, for example a catheter 36 or another component for delivering the combination of agent and fluid to a desired location within a body lumen of a patient. Delivery system 10 may include a valve assembly for selectively controlling the release of the powdered agent and/or the pressurized fluid within enclosure 14.

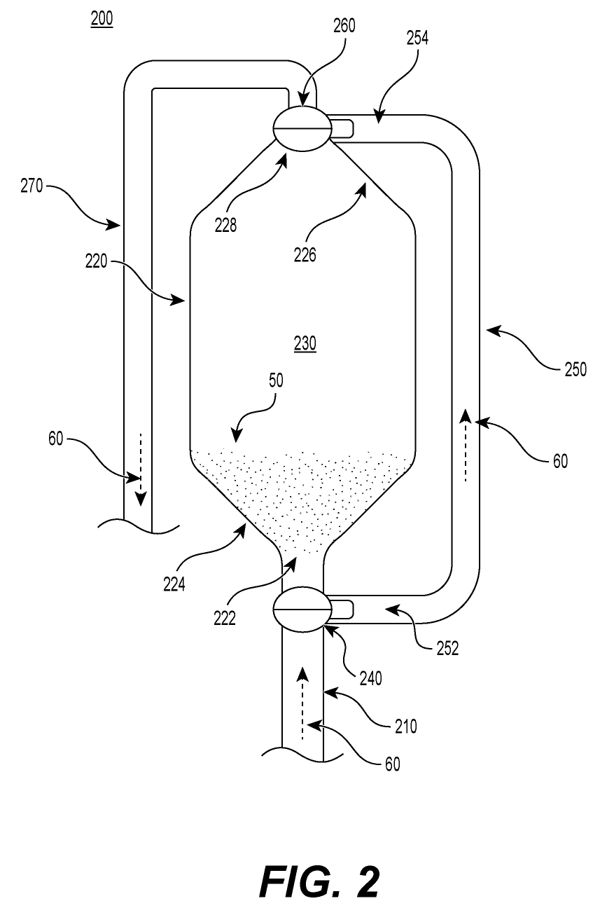

FIG. 2 shows an exemplary valve assembly 200, according to one or more embodiments. Valve assembly 200 may be part of delivery system 10 as discussed with reference to FIG. 1 above, such as fluidly coupled to enclosure 14. Valve assembly 200 may include an inlet 210 (e.g., a channel defining a lumen) in fluid communication with the pressurized fluid source (not shown) that is configured to store the pressurized fluid (e.g. gas, air, liquid, etc.), a chamber and/or enclosure 220 for storing a material, such as an agent 50 (e.g. a powdered agent), a first valve 240, a (bypass) channel 250, a second valve 260, and an outlet 270 (e.g., a tube defining a lumen) in fluid communication with a delivery conduit (e.g. catheter 36). In some embodiments, the fluid source may be external to delivery system 10 (FIG. 1), such that the fluid source may be fluidly coupled to delivery system 10, and particularly valve assembly 200. A predetermined amount of agent 50 may be stored within enclosure 220 for delivery from valve assembly 200. The predetermined amount of agent 50 stored therein may vary depending on an application of use, such as a treatment of a subject (e.g., patient) or a target treatment site. First valve 240 and second valve 260 may be sized, shaped, and/or otherwise configured to move, transition, and/or actuate to various different positions, states, and/or configurations as described in further detail herein. In some embodiments, one or more of valves 240, 260 may include a ball valve, a three-way stopcock valve, a luer valve, etc. In other embodiments, one or more of valves 240, 260 may be configured and operable as other suitable valving mechanisms without departing from a scope of this disclosure.

Enclosure 220 may be in fluid communication with inlet 210 and outlet 270 via first valve 240 and second valve 260, respectively. Enclosure 220 may include a first opening 222, a first funnel 224, a second funnel 226, and a second opening 228, which may collectively define a cavity 230 of enclosure 220 for storing the material, e.g., agent 50. First opening 222 may be positioned downstream from first funnel 224 of enclosure 220 for fluidly connecting cavity 230 of enclosure 220 to inlet 210 via first valve 240, such that enclosure 220 may be in fluid communication with inlet 210 at first opening 222. Second opening 228 may be positioned upstream from second funnel 226 of enclosure 220 for connecting enclosure 220 to outlet 270 via second valve 260, such that enclosure 220 may be in fluid communication with outlet 270 at second opening 228. One or more of first funnel 224 and/or second funnel 226 may be configured in a tapered, a conical, and/or an angled shape to guide agent 50 in moving within enclosure 220. In some examples, first funnel 224 may be configured to guide the pressurized fluid received in enclosure 220 via first opening 222 into cavity 230 and towards agent 50 stored therein to mix, combine, agitate, and/or otherwise fluidize agent 50 inside enclosure 220. In some examples, second funnel 226 may be configured to direct a mixture of the pressurized fluid and agent 50 out of enclosure 220 and towards second opening 228 for delivery of the mixture of the pressurized fluid and agent 50 towards outlet 270.

Channel 250 may be configured to receive the pressurized fluid received in valve assembly 200 at inlet 210. In some examples, channel 250 may be configured to allow the pressurized fluid to bypass enclosure 220 upon receipt within channel 250, such as based on a state of first valve 240 as described in further detail herein. Channel 250 may include a first end 252 and a second end 254 defining a longitudinal length between ends 252, 254. First end 252 may be positioned adjacent to first opening 222 and downstream from first opening 222 of enclosure 220. In this instance, first end 252 may be in fluid communication with inlet 210 via first valve 240. Second end 254 may be positioned adjacent to second opening 228 and upstream from second opening 228 of enclosure 220. In this instance, second end 254 may be in fluid communication with outlet 270 via second valve 260. It should be appreciated that channel 250, including each of first end 252 and second 254, may have various other suitable shapes, sizes, arrangements, and/or configurations than those shown and described herein without departing from a scope of this disclosure.

Still referring to FIG. 2, first valve 240 may be disposed downstream from one or more of enclosure 220 and/or channel 250. First valve 240 may be configured to selectively direct the pressurized fluid received from inlet 210 via the fluid source towards at least one of enclosure 220 and channel 250 based on a state and/or configuration of first valve 240. Second valve 260 may be disposed upstream from one or more of enclosure 220 and/or channel 250. Second valve 260 may be configured to selectively direct the pressurized fluid received from channel 250 via inlet 210 towards outlet 270 based on a state and/or configuration of second valve 260. Second valve 260 may be further configured to selectively direct the mixture of the pressurized fluid and agent 50 received from enclosure 220 towards outlet 270 and out of delivery system 10 based on the state and/or configuration of second valve 260.

In some embodiments, first valve 240 and second valve 260 may be configured to move simultaneously between a plurality of positions for selectively directing the pressurized fluid and/or agent 50 through valve assembly 200 via each of enclosure 220, channel 250, and/or both enclosure 220 and channel 250. In other embodiments, first valve 240 and second valve 260 may be configured to move independently from one another between the plurality of positions, respectively, for selectively delivering one or more of the pressurized fluid and/or agent 50. First valve 240 and/or second valve 260 may be configured to move in response to an actuation of delivery system 10, such as via actuation mechanism 30 (see FIG. 1). Although not shown, it should be appreciated that valves 240, 260 may be movably coupled to actuation mechanism 30, such as via one or more mechanical linkages.

In exemplary use, valve assembly 200 may be configured to selectively transition between a plurality of positions based on a state and/or configuration of one or more of first valve 240 and second valve 260. In some embodiments, valve assembly 200 may be configured to move towards a first position. When valve assembly 200 is in a first position, first valve 240 may be in a first state and/or configuration that is configured to close first opening 222 of enclosure 220 and open first end 252 of channel 250, while second valve 260 may be in a first state and/or configuration that is configured to close second opening 228 of enclosure 220 and open second end 254 of channel 250. In doing so, first valve 240 and second valve 260 may be configured to fluidly decouple enclosure 220 from inlet 210 and outlet 270, such that first valve 240 may be configured to inhibit the pressurized fluid in inlet 210 from entering enclosure 220 and second valve 260 may be configured to inhibit agent 50 (or a mixture of agent 50 and pressurized fluid within cavity 230) from exiting enclosure 220 through second opening 228.

Stated differently, enclosure 220 may not be in fluid communication with inlet 210 and outlet 270 when valve assembly 200 is in the first position and valves 240, 260 are in the first state and/or configuration, respectively. First valve 240 and second valve 260 may be configured to fluidly couple channel 250 with each of inlet 210 and outlet 270, such that first valve 240 may be configured to allow the pressurized fluid from inlet 210 to enter channel 250 through first end 252 and second valve 260 may be configured to allow the pressurized fluid to exit channel 250 and enter outlet 270 through second end 254. In other words, channel 250 may be in fluid communication with inlet 210 via first end 252 and outlet 270 via second end 254 when valve assembly 200 is in the first position. When enclosure 220 is decoupled from inlet 210 and outlet 270, first valve 240 may be configured to direct the pressurized fluid into channel 250 through first end 252 and away from the first opening 222 of enclosure 220 while second valve 260 may be configured to direct the pressurized fluid out of channel 250 and away from second opening 228 of enclosure 220, such that the channel 250 is configured to direct the pressurized fluid to bypass enclosure 220 for delivery of the pressurized fluid out of delivery system 10 in isolation from agent 50.

For example, still referring to FIG. 2, when valve assembly 200 is in the first position, a stream and/or flow 60 of the pressurized fluid may be received within inlet 210 and directed towards first valve 240, such as upon actuation of actuation mechanism 30 (see FIG. 1). Upon the pressurized fluid reaching first valve 240, flow 60 of the pressurized fluid may be directed into channel 250 via first valve 240, thereby bypassing enclosure 220, based on the state and/or configuration of first valve 240. Channel 250 may be configured to direct flow 60 of the pressurized fluid towards second end 254 until reaching second valve 260. Second valve 260 may be configured to direct flow 60 of the pressurized fluid from channel 250 towards outlet 270 and away from enclosure 220 based on the state and/or configuration of second valve 260.

In some embodiments, valve assembly 200 may be configured to move from the first position towards a second position that varies relative to the first position. When valve assembly 200 is in a second position, first valve 240 may be in a second state and/or configuration that is configured to open first opening 222 of enclosure 220 and close first end 252 of channel 250, while second valve 260 may be in a second state and/or configuration that is configured to open second opening 228 of enclosure 220 and close second end 254 of channel 250. First valve 240 and second valve 260 may be configured to fluidly couple enclosure 220 with each of inlet 210 and outlet 270, such that first valve 240 may be configured to allow the pressurized fluid to enter enclosure 220 from inlet 210 and second valve 260 may be configured to allow a mixture of agent 50 and the pressurized fluid to exit enclosure 220 via outlet 270. Stated differently, enclosure 220 may be in fluid communication with inlet 210 via first opening 222 and outlet 270 via second opening 228 when valve assembly 200 is in the second position with each of first valve 240 and second valve 260 in the second state and/or configuration, respectively.

Still referring to FIG. 2, first valve 20 and second valve 260 may be configured to fluidly decouple channel 250 from each of inlet 210 and outlet 270, such that first valve 240 may be configured to inhibit the pressurized fluid from entering first end 252 of channel 250 and second valve 260 may be configured to inhibit a remainder of the pressurized fluid received in channel 250 from exiting channel 250 via second end 254. In other words, channel 250 may not be in fluid communication with inlet 210 and outlet 270. First valve 240 may be configured to direct the pressurized fluid into enclosure 220 and away from channel 250, thereby moving, mixing, and/or agitating agent 50 stored within enclosure 220 with the pressurized fluid directed through first opening 222. In this instance, first valve 240 may be configured to generate a mixture of the pressurized fluid and agent 50 within cavity 230 of enclosure 220 while second valve 260 may be configured to release the mixture out of enclosure 220.

For example, when valve assembly 200 is in the second position, flow 60 of the pressurized fluid received via inlet 210 may be directed towards first valve 240. First valve 240 may be configured to direct flow 60 of the pressurized fluid into enclosure 220 via first opening 222. When first valve 240 is in the second state and/or configuration, flow 60 of the pressurized fluid is inhibited from entering channel 250. First funnel 224 may be configured to facilitate the pressurized fluid and agent 50 to mix within cavity 230 of enclosure 220, thereby creating a swirl or vortex of the mixture based on the angled and/or tapered configuration of first funnel 224. First valve 240 may be further configured to direct the mixture of pressurized fluid and agent 50 through cavity 230 and towards second opening 228 via second funnel 226. In this instance, second funnel 226 may be configured to guide the mixture from cavity 230 towards second opening 228 based on the angled and/or tapered configuration of second funnel 226. Second valve 260 may be configured to receive and direct the mixture of pressurized fluid and agent 50 out of enclosure 220 via second opening 228 and towards outlet 270.

In some embodiments, valve assembly 200 may be configured to move towards a third position that varies relative to the first and second positions. When valve assembly 200 is in a third position, first valve 240 may be in a third state and/or configuration that is configured to open each of first opening 222 of enclosure 220 and first end 252 of channel 250, and second valve 260 may be in a third state and/or configuration that is configured to open each of second opening 228 of enclosure 220 and second end 254 of the channel 250. In this instance, first valve 240 and second valve 260 may be configured to fluidly couple each of enclosure 220 and channel 250 with inlet 210 and outlet 270, such that first valve 240 may be configured to direct at least a portion of the pressurized fluid received from inlet 210 to enter each of enclosure 220 and channel 250, while second valve 260 may be configured to direct the mixture of agent 50 and pressurized fluid to exit enclosure 220 from second opening 228 and the pressurized fluid from channel 250 to exit second end 254. In this instance, valve assembly 200 may be configured to propel the mixture exiting enclosure 220 and entering outlet 270 with the portion of pressurized fluid received in channel 250 to facilitate delivery of the mixture through outlet 270 and out of valve assembly 200.

For example, still referring to FIG. 2, when valve assembly 200 is in the third position, first valve 240 may be configured to separate, divide, and/or distribute flow 60 of the pressurized fluid received from inlet 210 into multiple flow paths, such as towards both enclosure 220 and channel 250, respectively. In this instance, first valve 240 may be configured to direct a first portion of the pressurized fluid into enclosure 220 and a second portion of the pressurized fluid into channel 250. The first portion of the pressurized fluid may combine with agent 50 stored within cavity 230 of enclosure 220 while the second portion of the pressurized fluid may be directed through channel 250 towards second valve 260. Second valve 260 may be configured to direct the second portion of the pressurized fluid from channel 250 towards outlet 270.

Second valve 260 may be configured to direct the first portion of the pressurized fluid out of cavity 230 of enclosure 220 via second opening 228 and towards outlet 270. In this instance, valve assembly 200 may be configured to combine, group, and/or otherwise mix the combination of the first portion of pressurized fluid and agent 50 from enclosure 220 with the second portion of the pressurized fluid in isolation from channel 250 within outlet 270. Second valve 260 may be configured to deliver the second portion of the pressurized fluid for preventing clogging or a buildup of residual agent 50 within outlet 270 or the delivery conduit coupled thereto (e.g., catheter 36) before, during, and after delivery of the mixture of the pressurized fluid and agent 50. In other words, second valve 260 may be configured to provide insufflation through outlet 270 with the second portion of pressurized fluid received from channel 250, such as prior to receipt of the mixture from enclosure 220 into outlet 270, during simultaneous receipt of the mixture from enclosure 220 into outlet 270, and/or after completion of receipt of the mixture from enclosure 220 into outlet 270.

It should be appreciated that although valve assembly 200 is shown and described above as moving between three positions and each of valves 240, 260 moving between three states, respectively, valve assembly 200 and valves 240, 260 may be configured and operable to move towards additional and/or fewer positions and states without departing from a scope of this disclosure. Additionally, valve assembly 200 and valves 240, 260 may be configured and operable to move towards any one of the positions and/or states described above in various other sequences of operation than the arrangement described above without departing from a scope of this disclosure.

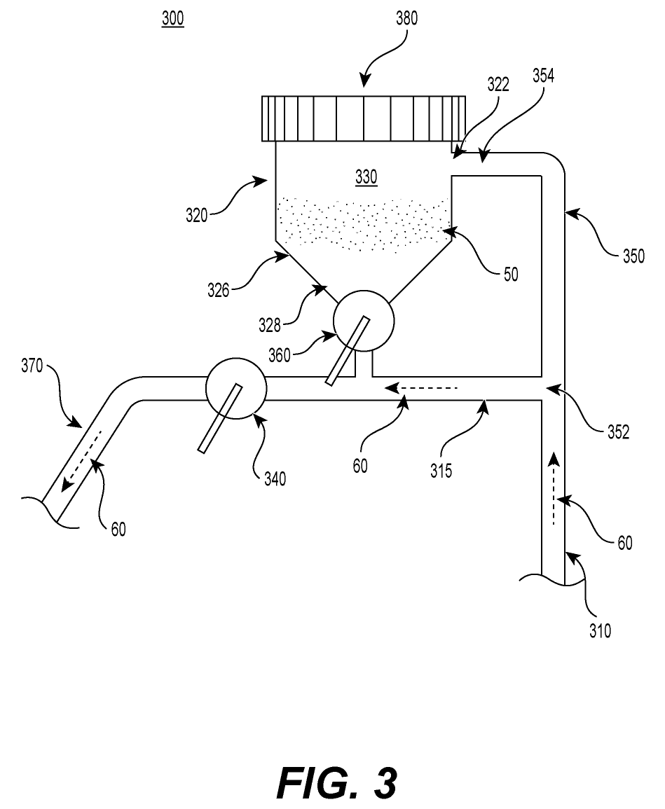

FIG. 3 shows another exemplary valve assembly 300, according to one or more embodiments. Valve assembly 300 may be part of delivery system 10 as discussed with reference to FIG. 1 above, such as fluidly coupled to enclosure 14. It should be appreciated that valve assembly 300 may be configured and operable like valve assembly 200 shown and described above except for the differences explicitly noted herein. Valve assembly 300 may include an inlet 310 in fluid communication with the pressurized fluid source (not shown) that is configured to store the pressurized fluid, a first channel 315 in fluid communication with inlet 310, a chamber and/or enclosure 320 for storing an agent 50, a first valve 340 disposed within first channel 315, a second channel 350 in fluid communication with inlet 310, a second valve 360, an outlet 370 in fluid communication with a delivery conduit (e.g., catheter 36), and a cap 380 removably coupled to enclosure 320. A predetermined amount of agent 50 may be stored within enclosure 320 for delivery from valve assembly 300. The predetermined amount of agent stored therein may vary depending on a desired application of use, such as a treatment of a subject (e.g., patient) or a target treatment site. Valves 340, 360 may be configured and operable in a substantially similar manner as valves 240, 260 shown and described above.

Enclosure 320 may be in fluid communication with inlet 310 and outlet 370 via first valve 340 and second valve 360, respectively. Enclosure 320 may include a first opening 322, a funnel 326, and a second opening 328, which may collectively define a cavity 330 of enclosure 320 for storing a material (e.g., powdered agent). First opening 322 may be positioned downstream from second channel 350, thereby connecting enclosure 320 to inlet 310 via second channel 350. First opening 322 may be in fluid communication with inlet 310 via second channel 350. Second opening 328 may be positioned downstream from funnel 326 of enclosure 320, thereby connecting enclosure 320 to outlet 370 via second valve 360 disposed therebetween. Funnel 326 may be configured in a conical and/or angled shape to assist agent 50 in moving within and/or exiting enclosure 320 via second opening 328. For example, funnel 326 may be configured to direct a mixture of the pressurized fluid and agent 50 out of enclosure 220 and towards second opening 328 for delivery of the mixture of the pressurized fluid and agent 50 towards outlet 270. Cap 380 may be rotatably coupled to enclosure 320 for sealing cavity 330. Cap 380 may be secured to enclosure 320 by one or more threads (not shown) or other securing mechanisms. In some embodiments, cap 380 may be selectively attachable to enclosure 320 to facilitate receipt and/or refill of agent 50 within cavity 330 during subsequent uses of delivery system 10.

Still referring to FIG. 3, first channel 315 may be configured to receive a first portion of a pressurized fluid from the pressurized fluid source via inlet 310. Second channel 350 may be configured to receive a second portion of the pressurized fluid from the pressurized fluid source via inlet 310. In some embodiments, second channel 350 may be in fluid communication with enclosure 320 at first opening 322, such that second channel 350 may be configured to deliver the second portion of the pressurized fluid into enclosure 320 for mixing with agent 50 in cavity 330. Valve assembly 300 may be sized, shaped, and/or otherwise configured to separate the pressurized fluid received from inlet 310 into the first portion of pressurized fluid and the second portion of the pressurized fluid at a first end 352 of second channel 350. First end 352 of second channel 350 may be configured to direct the first portion of pressurized fluid through first channel 315 and towards outlet 370, and direct the second portion of pressurized fluid through second channel 350 and towards a second end 354 of second channel 350. Second end 354 of second channel 350 may be fluidly coupled to enclosure 320 via first opening 322. The second portion of pressurized fluid may be directed into enclosure 320 by second end 354 and through first opening 322 via second channel 350.

In some embodiments, first valve 340 may be disposed within first channel 315 downstream from enclosure 320. First valve 340 may be configured to selectively direct the first portion of the pressurized fluid received in first channel 315 towards outlet 370 while bypassing enclosure 320 based on a state and/or configuration of first valve 340. Second valve 360 may be disposed downstream from enclosure 320 and upstream from first channel 315. Second valve 360 may be configured to selectively direct a mixture of the second portion of the pressurized fluid received within cavity 330 from second channel 350 with agent 50 stored in enclosure 320 into first channel 315 based on a state and/or configuration of second valve 360. In some embodiments, first valve 340 and second valve 360 may be configured to move simultaneously between a plurality of positions for selectively directing a mixture of the pressurized fluid and agent 50 through the delivery system 10 via one or more of first channel 315, second channel 350, and/or enclosure 320. In other embodiments, first valve 340 and second valve 360 may be configured to move independently from one another between the plurality of positions, respectively, for selectively delivering one or more of the pressurized fluid and/or agent 50. First valve 340 and/or second valve 360 may be configured to move in response to an actuation of delivery system 10, such as via actuation mechanism 30 (see FIG. 1).

In exemplary use, valve assembly 300 may be configured to selectively transition between a plurality of positions based on a state and/or configuration of one or more of first valve 340 and second valve 360. In some embodiments, valve assembly 300 may be configured to move towards a first position. When valve assembly 300 is in a first position, first valve 340 may be in a first state that is configured to direct the first portion of the pressurized fluid received in first channel 315 from inlet 310 towards outlet 370 to exit delivery system 10, while second valve 360 may be in a first state that is configured to inhibit a mixture of agent 50 and the second portion of the pressurized fluid received in enclosure 320 from second channel 350 from entering first channel 315. In doing so, inlet 310 may be in fluid communication with outlet 370 via first channel 315 through first valve 340. In addition, second valve 360 may be configured to inhibit the first portion of pressurized fluid received in first channel 315 from entering enclosure 320 via second opening 328.

For example, when assembly 300 is in the first position, first end 352 may be sized, shaped, and/or otherwise configured to direct flow 60 of the pressurized fluid within inlet 310 towards each of first channel 315 and second channel 350. Upon reaching first end 352 of second channel 350, first end 352 may be configured to separate, divide, and/or otherwise distribute the pressurized fluid into the first portion and the second portion, respectively, where the first portion is directed towards outlet 370 through first channel 315 and the second portion is directed towards first opening 322 of enclosure 320 through second channel 350.

Second channel 350 may be configured to guide the second portion of pressurized fluid to enter enclosure 320 through first opening 322 disposed adjacent to second end 354 of second channel 350, thereby agitating agent 50 stored therein. Second channel 350 may be configured to create a mixture of the pressurized fluid and agent 50 within enclosure 320 in response to guiding the second portion of pressurized fluid into cavity 330 via first opening 322. Second channel 350 may be configured to facilitate the second portion of the pressurized fluid creating a swirl or vortex within cavity 330 for agitating agent 50 stored within enclosure 320, thereby assisting in mixing the second portion of pressurized fluid and agent 50. In some examples, second end 354 of second channel 350 may be sized, shaped, and/or otherwise configured in an orientation and/or position relative to first opening 322 to urge the second portion of pressurized fluid into enclosure 320 at an angle and/or arrangement that facilitates creation of the swirl or vortex in cavity 330.

Still referring to FIG. 3, second end 354 of second channel 350 may be positioned relative to an upper portion/end of enclosure 320 adjacent to cap 380 to assist in agitating agent 50 stored within cavity 330 towards a lower end and/or intermediate portion of enclosure 320. In this instance, the second portion of pressurized fluid enters enclosure 320 relatively above agent 50. Second valve 360 may be configured to inhibit the mixture from exiting enclosure 320 through second opening 328 and into first channel 315 based on the second state of second valve 360. In this instance, first valve 340 may be configured to provide insufflation through outlet 370 with the first portion of pressurized fluid received from first channel 350, such as prior to receipt of the mixture from enclosure 320 into first channel 315 via second valve 360.

In some embodiments, valve assembly 300 may be configured to move from the first position towards a second position that varies relative to the first position. When valve assembly 300 is in a second position, second valve 360 may be in a second state that is configured to release the mixture in enclosure 320 into first channel 315. Additionally, first valve 340 may be in a second state that is configured to direct the mixture received within first channel 315 via second valve 360 and the first portion of the pressurized fluid towards outlet 370. In this instance, enclosure 320 may be in fluid communication with inlet 310 via second channel 350, and outlet 370 via first valve 340 and second valve 360 through first channel 315.

For example, when valve assembly 300 is in the second position, flow 60 of the pressurized fluid received via inlet 310 may be directed towards first end 352 of second channel 350. First end 352 may be configured to separate flow 60 of the pressurized fluid into the first portion of pressurized fluid and the second portion of pressurized fluid as described above. The first portion of pressurized fluid may be directed towards first channel 315 and outlet 370, while the second portion of pressurized fluid may be directed through second channel 350 and towards first opening 322 of enclosure 320 that is disposed adjacent to second end 354. The second portion of pressurized fluid may enter first opening 322 and mix with agent 50 stored therein. Based on a position, arrangement, and/or orientation of second end 354 relative to first opening 322 of enclosure 320, second channel 350 may be configured to direct the second portion of pressurized fluid into cavity 330 to urge the mixture formed within enclosure 320 towards second opening 328 via funnel 326.

Upon the mixture exiting enclosure 320 through second opening 328 via second valve 360, the first portion of pressurized fluid within first channel 315 may direct the mixture towards outlet 370. First valve 340 may be configured to permit the mixture and the first portion of pressurized fluid to exit valve assembly 300 via outlet 370 for delivery from delivery system 10. In other words, first valve 340 may be configured to provide insufflation through outlet 370 with the first portion of pressurized fluid received from first channel 315, such as prior to receipt of the mixture from enclosure 320 into first channel 315, during simultaneous receipt of the mixture from enclosure 320 into first channel 315, and/or after completion of receipt of the mixture from enclosure 320 into first channel 315.

It should be appreciated that although valve assembly 300 is shown and described above as moving between two positions and each of valves 340, 360 moving between two states, respectively, valve assembly 300 and valves 340, 360 may be configured and operable to move towards additional and/or fewer positions and states without departing from a scope of this disclosure. Additionally, valve assembly 300 and valves 340, 360 may be configured and operable to move towards any one of the positions and/or states described above in various other sequences of operation than the arrangement described above without departing from a scope of this disclosure.

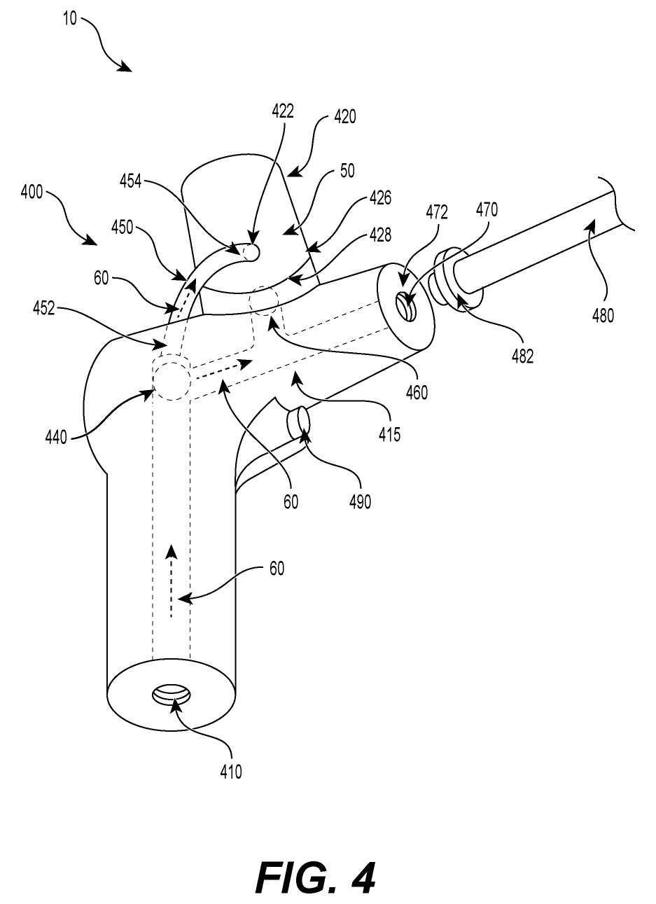

FIG. 4 shows another exemplary valve assembly 400, according to one or more embodiments. Valve assembly 400 may be part of delivery system 10 as discussed with reference to FIG. 1 above, such as fluidly coupled to enclosure 14. It should be appreciated that valve assembly 400 may be configured and operable like valve assemblies 200, 300 shown and described above except for the differences explicitly noted herein. Valve assembly 400 may include an inlet 410 in fluid communication with the pressurized fluid source (not shown) that is configured to store the pressurized fluid, a first channel 415 in fluid communication with inlet 410, a chamber and/or enclosure 420 for storing agent 50, a first valve 440 disposed at least partially within inlet 410, a second channel 450 in fluid communication with inlet 410 via first valve 440, a second valve 460 in fluid communication with enclosure 420, an outlet 470 in fluid communication with a delivery conduit 480, and an actuation mechanism 490. A predetermined amount of agent 50 may be stored within enclosure 420 for delivery from valve assembly 400. The predetermined amount of agent 50 stored therein may vary depending on a desired application of use, such as a treatment of a subject (e.g., patient) or a target treatment site.

It should be appreciated that delivery conduit 480 may be configured and operable similar to delivery conduit 36, actuation mechanism 490 may be configured and operable similar to actuation mechanism 30, as described above except for the differences explicitly noted herein, respectively. Enclosure 420 may be in fluid communication with inlet 410 and outlet 470 via first valve 440 and second valve 460, respectively. Enclosure 420 may include a first opening 422, a funnel 426, and a second opening 428, which may collectively define a cavity within enclosure 420 for storing a material (e.g., powdered agent). First opening 422 may be positioned downstream from second channel 450, thereby fluidly connecting enclosure 420 to inlet 410 via second channel 450. First opening 422 may be in fluid communication with inlet 410 via second channel 450. Second opening 428 may be positioned downstream from funnel 426 of enclosure 420, thereby fluidly connecting enclosure 420 to outlet 470 via second valve 460 that is disposed adjacent to second opening 428. Funnel 426 may be configured in a conical and/or angled shape to guide agent 50 stored within enclosure 420 towards second opening 428. For example, funnel 426 may be configured to direct agent 50 towards second opening 428 during delivery of a mixture of the pressurized fluid and agent 50 out of enclosure 420. Outlet 470 may include a fastening member 472 that may be configured to be rotatably fastened to delivery conduit 480 (e.g., a catheter) via a fastening member 482. Fastening members 472 and 482 may include various suitable fastening mechanisms configured to fluidly connect outlet 470 and delivery conduit 480 to one another. For example, fastening members 472, 482 may include one or more threads, a male/female luer connector, or other suitable securing mechanisms.

Inlet 410 may be configured to receive the pressurized fluid from the pressurized fluid source (not shown). Inlet 410 may be in selective fluid communication with first channel 415 and second channel 450 via first valve 440 disposed therebetween based on a state and/or configuration of first valve 440. When inlet 410 is in fluid communication with first channel 415, first valve 440 may be configured to direct the pressurized fluid received in inlet 410 towards first channel 415 to bypass enclosure 420 and enter outlet 470. When inlet 410 is in fluid communication with second channel 450, first valve 440 may be configured to direct the pressurized fluid received in inlet 410 towards enclosure 420 to combine or mix with agent 50 stored therein. When inlet 410 is in fluid communication with both first channel 415 and second channel 450, first valve 440 may be configured to direct the pressurized fluid received in inlet 410 into separate flow paths, such as into a first portion of flow 60 of pressurized fluid and a second portion of flow 60 of pressurized fluid. The first portion of pressurized fluid may be directed towards outlet 470 through first channel 415 while the second portion of pressurized fluid may be directed into enclosure 420 through second channel 450.

First channel 415 may be configured to receive the pressurized fluid received in inlet 410 via first valve 440. First channel 415 may be disposed between first valve 440 and outlet 470. Second channel 450 may be configured to receive the pressurized fluid received in inlet 410 via first valve 440. Second channel 450 may include a first end 452 disposed adjacent to and in fluid communication with first valve 440, and a second end 454 disposed adjacent to and in fluid communication with first opening 422 of enclosure 420.

In some embodiments, first valve 440 may be disposed upstream from enclosure 420 and first channel 415 and second channel 450. First valve 440 may be configured to selectively direct the pressurized fluid towards at least one of enclosure 420 through second channel 450 and outlet 470 through first channel 415. Second valve 460 may be disposed downstream from enclosure 420. Second valve 460 may be configured to selectively direct a mixture of the pressurized fluid and agent 50 out of enclosure 420 and into first channel 415. In some embodiments, first valve 440 and second valve 460 may be configured to move simultaneously between a plurality of positions for selectively directing a mixture of the pressurized fluid and agent 50 through the delivery system 10 via one or more of first channel 415, second channel 450, and/or enclosure 420. In other embodiments, first valve 440 and second valve 460 may be configured to move independently from one another between the plurality of positions, respectively, for selectively delivering one or more of the pressurized fluid and/or agent 50. First valve 440 and/or second valve 460 may be configured to move in response to an actuation of delivery system 10, such as via actuation mechanism 490, discussed in further detail below.

In exemplary use, valve assembly 400 may be configured to selectively transition between a plurality of positions based on a state and/or configuration of one or more of first valve 440 and second valve 460. In some embodiments, valve assembly 400 may be configured to move towards a first position, and first valve 440 may be in a first state that is configured to direct the pressurized fluid towards outlet 470 through first channel 415 to exit delivery system 10, while second valve 460 may be in a first state that is configured to inhibit a mixture of agent 50 and pressurized fluid received in enclosure 420 from entering first channel 415. In doing so, inlet 410 may be in fluid communication with outlet 470 via first channel 415 through first valve 440 allowing the pressurized fluid to bypass enclosure 420. In addition, second valve 460 may be configured to inhibit the pressurized fluid received in first channel 415 from entering enclosure 420 via second opening 428. First valve 440 and second valve 460 may be configured to fluidly couple first channel 415 and second channel 450 with inlet 410 and outlet 470, respectively. First valve 440 may be configured to allow the pressurized fluid to enter first channel 415 and be directed towards outlet 470, and second valve 460 may be configured to allow the pressurized fluid to exit enclosure 420 through second opening 428 and enter first channel 415.

For example, when valve assembly is in the first position, flow 60 of the pressurized fluid may be received within inlet 410 and directed towards first valve 440. Upon reaching first valve 440, first valve 440 may be configured to direct flow 60 of the pressurized fluid into first channel 415, thereby bypassing enclosure 420. First valve 440 may be configured to guide flow 60 of the pressurized fluid through first channel 415 and towards outlet 470.

When valve assembly 400 is in a second position, first valve 440 may be configured to allow the pressurized fluid to enter enclosure 420 to generate a mixture with agent 50, while second valve 460 may be configured to allow the mixture to exit enclosure 420 and enter into first channel 415 for delivery via outlet 470. Inlet 410 may be in fluid communication with enclosure 420 via second channel 450 when first valve 440 is in the second state, allowing the pressurized fluid to enter into enclosure 420 to generate the mixture of pressurized fluid and agent 50 stored therein. In this instance with first valve 440 in the second state, inlet 410 may not be in fluid communication with outlet 470 via first channel 415. First valve 440 and second valve 460 may be configured such that inlet 410 may be in fluid communication with outlet 470 via second channel 450 and enclosure 420. The mixture of pressurized fluid and agent 50 may exit enclosure 420 and be directed towards outlet 470 for delivery when first valve 440 and second valve 460 are in the second state.

For example, when valve assembly 400 is in the second position, flow 60 of the pressurized fluid received via inlet 410 may be directed towards first valve 440. First valve 440 may be configured to direct flow 60 of pressurized fluid into first opening 422 of enclosure 420 through second channel 450. First opening 422 may be disposed adjacent to second end 454 of second channel 450, such that the pressurized fluid received therein may agitate the agent stored within enclosure 420. When first valve 440 is in the second position, flow 60 of pressurized fluid in inlet 410 may be prohibited from entering first channel 415 by first valve 440.

Funnel 426 may be configured to facilitate a mixture of the pressurized fluid and agent 50 within cavity 430 of enclosure 420 by creating a swirl or vortex based on the angle and/or tapered profile of funnel 426. Second channel 450 may be sized, shaped, and/or otherwise configured to direct the pressurized fluid into enclosure 420 such that the mixture of pressurized fluid and agent 50 in enclosure 420 may be directed towards second opening 428 via funnel 426. Second valve 460 may be configured to permit the mixture of pressurized fluid and agent 50 to exit enclosure 420 via second opening 428 and enter into first channel 415 for delivery towards outlet 470. Outlet 470 may be configured to couple with delivery conduit 480, such that the mixture may be delivered out of delivery system 10 via delivery conduit 480.

In some embodiments, valve assembly 400 may be configured to move towards a third position that varies relative to the first and second positions. When valve assembly 400 is in a third position, first valve 440 may be in a third state and/or configuration that is configured to open each of first end 452 of second channel 450 and first channel 415, and second valve 460 may be in a third state and/or configuration that is configured to open the second opening 428 of enclosure 420. In this instance first valve 440 and second valve 460 may be configured to fluidly couple each of first channel 415, enclosure 420, and second channel 450 with inlet 410 and outlet 470. For example, first valve 440 may be configured to direct at least a portion of the pressurized fluid received in inlet 410 towards each of first channel 415 and second channel 450, while second valve 460 may be configured to direct the mixture of agent 50 and pressurized fluid out of enclosure 420 via second opening 428. In this instance, first valve 440 may be configured to direct the pressurized fluid from first channel 415 to deliver the mixture received from enclosure 420 via second opening 428 towards the outlet 470. In other words, valve assembly 400 may be configured to propel the mixture exiting enclosure 420 and entering outlet 470 with the portion of pressurized fluid received in first channel 415 to facilitate delivery of the mixture through outlet 470 and out of valve assembly 400.

For example, still referring to FIG. 4, when valve assembly 400 is in the third position, first valve 440 may be configured to separate, divide, and/or distribute flow 60 of the pressurized fluid received from inlet 410 into multiple flow paths, such as towards both first channel 415 and enclosure 420, respectively. In this instance, first valve 440 may be configured to direct a first portion of pressurized fluid into first channel 415 and a second portion of pressurized fluid into second channel 450. The second portion of pressurized fluid may combine with agent 50 stored within the cavity of enclosure 420 while the first portion of pressurized fluid may be directed through first channel 415 and towards outlet 470 to facilitate delivery of the mixture received from enclosure 420.

Second valve 460 may be configured to direct the mixture of pressurized fluid and agent 50 to out of the cavity of enclosure 420 via second opening 428 and towards outlet 470 via first channel 415. In this instance, valve assembly 400 may be configured to combine, group, and/or otherwise mix the combination of the first portion of pressurized fluid with the second portion of pressurized fluid and agent 50 from enclosure 420 within outlet 470. First valve 440 may be configured to deliver the first portion of the pressurized fluid for preventing clogging or a buildup of residual agent 50 within outlet 470 or delivery conduit 480 coupled thereto before, during, and/or after delivery of the mixture of pressurized fluid and agent 50. In other words, first valve 440 may be configured to provide insufflation through outlet 470 with the first portion of pressurized fluid received from first channel 415, such as prior to receipt of the mixture from enclosure 420 into outlet 470, during simultaneous receipt of the mixture from enclosure 420 into outlet 470, and/or after completion of receipt of the mixture from enclosure 420 into outlet 470.

Actuation mechanism 490 may be configured to actuate first valve 440 and second valve 460 simultaneously into the plurality of positions. Actuation mechanism 490 may be a trigger as shown in FIG. 4, however, additional actuation mechanisms may be used in place of or with actuation mechanism 490. For example, actuation mechanism 490 may be pulled or pressed such that first valve 440 and second valve 460 may move into at least the first position, the second position, and the third position. In some embodiments, actuation mechanism 490 may be configured to be pulled by a first amount and/or extent of a movement stroke to move first valve 440 and second valve 460 towards the first state. Actuation mechanism 490 may be further configured to be pulled a second amount and/or extent of a movement stroke from and subsequent to the first amount of movement stroke, to move first valve 440 and second valve 460 towards the second state. In other words, a single actuation of actuation mechanism 490 may be configured to move first valve 440 and second valve 460 to multiple states and/or positions based on an extent or amount of movement of actuation mechanism 490.

In some embodiments, actuation mechanism 490 may be configured to be pulled a third amount and/or extent of a movement stroke that is greater than and subsequent to the first and second amounts. In this instance, actuation mechanism 490 may be configured to move first valve 440 and second valve 460 towards a third state. Upon commencing release of actuation mechanism 490, first valve 440 and second valve 460 may initially return to the first state, thereby allowing for the pressurized fluid received via inlet 410 to flow through first channel 415 towards outlet 470. Upon full release of actuation mechanism 490, first valve 440 and second valve 460 may move to a fully closed position in which the pressurized fluid received in inlet 410 is inhibited from entering first channel 415 and second channel 450. Although actuation mechanism 490 is described within FIG. 4, it should be appreciated that actuation mechanism 490 may be incorporated with each of valve assemblies 200, 300 shown and described above.

It should be appreciated that although valve assembly 400 is shown and described above as moving between three positions and each of valves 440, 460 moving between three states, respectively, valve assembly 400 and valves 440, 460 may be configured and operable to move towards additional and/or fewer positions and states without departing from a scope of this disclosure. Additionally, valve assembly 400 and valves 440, 460 may be configured and operable to move towards any one of the positions and/or states described above in various other sequences of operation than the arrangement described above without departing from a scope of this disclosure.

Although valve assemblies 200, 300, and 400 are shown and described herein as being implemented in delivery system 10, and particularly in a handheld device, it should be appreciated that they may be configured and operable in various other suitable configurations. For example, valve assemblies 200, 300, and 400 may be directly coupled to a delivery device (e.g., an endoscope, a catheter, etc.) that is positioned adjacent to a target treatment site within a subject (e.g., a patient).

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description. The features described herein may be used alone or in combination and are not mutually exclusive.

What is claimed is:

1. A device for delivering an agent, comprising:
an enclosure configured to store the agent;
a channel configured to receive a pressurized fluid, wherein the channel bypasses the enclosure;
a first valve disposed downstream from the enclosure and the channel, wherein the first valve is configured to selectively direct the pressurized fluid towards at least one of the enclosure and the channel; and
a second valve disposed upstream from the enclosure and the channel, wherein the second valve is configured to selectively direct the pressurized fluid received from the channel and the agent received from the enclosure out of the device for delivery;
wherein the first valve and the second valve are configured to move simultaneously between a plurality of positions for selectively directing the pressurized fluid through the device via each of the channel, the enclosure, and both of the channel and the enclosure.

2. The device of claim 1, further comprising:
an inlet in fluid communication with a fluid source that is configured to store the pressurized fluid; and
an outlet in fluid communication with a delivery conduit.

3. The device of claim 2, wherein the enclosure includes a first opening in fluid communication with the inlet and a second opening in fluid communication with the outlet.

4. The device of claim 3, wherein the channel includes a first end positioned adjacent to the first opening and in fluid communication with the inlet, and a second end positioned adjacent to the second opening and in fluid communication with the outlet.

5. The device of claim 4, wherein when in a first position, the first valve is configured to close the first opening of the enclosure and open the first end of the channel while the second valve is configured to close the second opening of the enclosure and open the second end of the channel.

6. The device of claim 5, wherein when in the first position, the first valve and the second valve are configured to fluidly decouple the enclosure from the inlet and the outlet, such that the first valve is configured to inhibit the pressurized fluid from entering the enclosure and the second valve is configured to inhibit the agent from exiting the enclosure.

7. The device of claim 6, wherein when in the first position, the first valve and the second valve are configured to fluidly couple the channel with the inlet and the outlet, such that the first valve is configured to allow the pressurized fluid to enter the channel and the second valve is configured to allow the pressurized fluid to exit the channel.

8. The device of claim 5, wherein when in a second position, the first valve is configured to open the first opening of the enclosure and close the first end of the channel while the second valve is configured to open the second opening of the enclosure and close the second end of the channel.

9. The device of claim 8, wherein when in the second position, the first valve and the second valve are configured to fluidly couple the enclosure with the inlet and the outlet, such that the first valve is configured to allow the pressurized fluid to enter the enclosure and the second valve is configured to allow a mixture of the agent and the pressurized fluid to exit the enclosure.

10. The device of claim 8, wherein when in the second position, the first valve and the second valve are configured to fluidly decouple the channel from the inlet and the outlet, such that the first valve is configured to inhibit the pressurized fluid from entering the channel and the second valve is configured to inhibit a remainder of the pressurized fluid from exiting the channel.

11. The device of claim 8, wherein when in a third position, the first valve is configured to open the first opening of the enclosure and the first end of the channel, and the second valve is configured to open the second opening of the enclosure and the second end of the channel.

12. The device of claim 11, wherein when in the third position, the first valve and the second valve are configured to fluidly couple each of the enclosure and the channel with the inlet and the outlet, such that the first valve is configured to allow the pressurized fluid to enter the enclosure and the channel while the second valve is configured to allow a mixture of the agent and the pressurized fluid to exit the enclosure and the pressurized fluid from the channel to deliver the mixture towards the outlet.

13. The device of claim 1, wherein when in a first position, the first valve is configured to direct the pressurized fluid into the channel and away from the enclosure while the second valve is configured to direct the pressurized fluid out of the channel and away from the enclosure, such that the pressurized fluid bypasses the enclosure for delivery in isolation from the agent.

14. The device of claim 13, wherein when in a second position, the first valve is configured to direct the pressurized fluid into the enclosure and away from the channel, thereby agitating the agent stored within the enclosure with the pressurized fluid.

15. The device of claim 14, wherein when in the second position, the first valve is configured to generate a mixture of the pressurized fluid and the agent within the enclosure while the second valve is configured to release the mixture out of the enclosure.

16. A device for delivering an agent, comprising:
an enclosure configured to store the agent;
a first channel configured to receive a first portion of a pressurized fluid;
a second channel configured to receive a second portion of the pressurized fluid, wherein the second channel is in fluid communication with the enclosure such that the second channel is configured to deliver the second portion of the pressurized fluid into the enclosure for mixing with the agent;
a first valve disposed within the first channel and downstream from the enclosure, wherein the first valve is configured to selectively direct the first portion of the pressurized fluid received in the first channel out of the device for delivery; and
a second valve disposed downstream from the enclosure and upstream from the first channel, wherein the second valve is configured to selectively direct the second portion of the pressurized fluid received from the second channel and the agent received from the enclosure into the first channel;
wherein the first valve and the second valve are configured to move simultaneously between a plurality of positions for selectively directing the pressurized fluid through the device via one or more of the first channel, the second channel, and the enclosure.

17. The device of claim 16, wherein when in a first position, the first valve is configured to allow the first portion of the pressurized fluid received in the first channel to exit the device for delivery while the second valve is configured to inhibit a mixture of the agent and the second portion of the pressurized fluid received in the enclosure from the second channel from entering the first channel.

18. The device of claim 17, wherein when in a second position, the first valve is configured to allow the mixture to enter the first channel and the first portion of the pressurized fluid to direct the mixture out of the first channel out of the device for delivery.

19. A device for delivering an agent, comprising:
an enclosure configured to store the agent;
a channel configured to receive a pressurized fluid, wherein the channel bypasses the enclosure;
a first valve disposed upstream from the enclosure and the channel, wherein the first valve is configured to selectively direct the pressurized fluid towards at least one of the enclosure and the channel; and
a second valve disposed downstream from the enclosure, wherein the second valve is configured to selectively direct the agent out of the enclosure and into the channel;
wherein the first valve and the second valve are configured to move simultaneously between a plurality of positions for selectively directing the pressurized fluid through the device via each of the channel and the enclosure.

20. The device of claim 19, wherein when in a first position, the first valve is configured to allow the pressurized fluid to enter the channel and exit the device for delivery while the second valve is configured to inhibit the agent from exiting the enclosure into the channel; and
wherein in a second position, the first valve is configured to allow the pressurized fluid to enter the enclosure to generate a mixture with the agent while the second valve is configured to allow the mixture to exit the enclosure into the channel for delivery out of the device.
